(19) United States
(12) Patent Application Publication
(10) Pub. No.: US 2026/0315810 A1
(43) Pub. Date: Oct. 15, 2026

(54) COMPACT ENERGY HARVESTERS FOR IMPLANTABLE DEVICES

(71) Applicant: Medtronic, Inc., Minneapolis, MN (US)

(72) Inventors: Christian S. Nielsen, River Falls, WI (US); Amin Karami, Ann Arbor, MI (US); Anthony W. Olivett, Brooklyn Center, MN (US)

(21) Appl. No.: 19/485,441

(22) PCT Filed: May 10, 2024
(86) PCT No.: PCT/IB2024/054558

§ 371 Date: Nov. 18, 2025

Related U.S. Application Data
(60) Provisional application No. 63/503,372, filed on May 19, 2023.

(51) Int. Cl. A61N 1/372; A61N 1/378; H02J 50/00; H02J 105/46; H02N 2/18

(52) U.S. Cl. CPC A61N 1/37205; A61N 1/3785; H02J 50/001; H02J 50/005; H02N 2/188; H02J 2105/46

(57) ABSTRACT

Energy harvesting mechanisms for implantable devices are provided. In some embodiments, a device includes a housing configured to be implanted within a patient. The device can include a power assembly positioned within the housing and having a power source, and an energy harvesting mechanism positioned within the housing and configured to charge the power source. The energy harvesting mechanism can include: a first piezoelectric element coupled to a fixation region within the housing, a second piezoelectric element coupled to a harvester mass, and a third piezoelectric element coupled in series between the first and second piezoelectric elements. The second and third piezoelectric elements can be positioned at different sides of the first piezoelectric element.

This application claims the benefit of U.S. Provisional Patent Application Ser. No. 63/503,372, filed May 19, 2023, the entire content of which is incorporated herein by reference.

### TECHNICAL FIELD

The present technology generally relates to medical devices, and in particular, to compact energy harvesters for implantable devices.

### BACKGROUND

Various types of implantable medical devices have been developed for monitoring or treating one or more conditions of a patient. For example, a cardiac pacemaker can monitor a patient's heart activity and provide therapeutic electrical stimulation to the heart via electrodes. The electrical stimulation provided by the cardiac pacemaker can include signals such as pacing pulses to address abnormal cardiac rhythms (e.g., bradycardia). Some types of cardiac pacemakers are implanted a distance from the heart and are coupled to one or more leads that extend intravascularly into the heart to position the electrodes in contact with cardiac tissue. However, the leads may be prone to fracture, which may result in unreliable or incorrect pacing, and may require replacement of the lead or even the entire pacemaker.

Some types of cardiac pacemakers are sized to be completely implanted within one of the chambers of the heart, and may include electrodes integrated with or attached to the device housing rather than leads. Such pacemakers can be less invasive than traditional pacemakers and can avoid complications associated with lead fracture. However, the relatively small size of such pacemakers may limit the types of power sources that can be incorporated into the device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a pacing device implanted in the heart of a patient, in accordance with embodiments of the present technology.

FIG. 2 is a perspective view of a pacing device configured in accordance with embodiments of the present technology.

FIG. 3 is a side view of another pacing device configured in accordance with embodiments of the present technology.

FIG. 4 is a schematic block diagram illustrating electronic components of a pacing device configured in accordance with embodiments of the present technology.

FIG. 5A is a perspective view of a device including an energy harvesting mechanism, in accordance with embodiments of the present technology.

FIG. 5B is a cross-sectional view of the device of FIG. 5A.

FIG. 6 is a perspective view of an energy harvesting mechanism, in accordance with the embodiments of the present technology.

### DETAILED DESCRIPTION

The present technology relates to energy harvesters for implantable devices. In some embodiments, for example, a device includes a housing configured to be implanted within a patient. The device can also include a power assembly positioned within the housing and including a power source. The power assembly can be a tubular structure having a lumen extending therethrough. The device can further include an energy harvesting mechanism positioned within the housing and configured to charge the power source. The energy harvesting mechanism can include a first piezoelectric element coupled to a fixation region within the housing, a second piezoelectric element coupled to a harvester mass, and at least one third piezoelectric element coupled in series between the first and second piezoelectric elements. In some embodiments, the second and third piezoelectric elements are positioned at different sides of the first piezoelectric element, such that the first piezoelectric element overlaps (e.g., is coaxial with) a central longitudinal axis of the housing and/or the power assembly. This configuration allows at least a portion of the first piezoelectric element to be received within the lumen of the power assembly, which can reduce the overall space requirements for the energy harvesting mechanism while also maintaining a low resonant frequency compatible with the frequencies present in physiological motion, thus allowing the energy harvesting mechanism to be used in implantable devices with strict size constraints (e.g., devices implanted in a single heart chamber).

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Overview of Implantable Pacing Devices

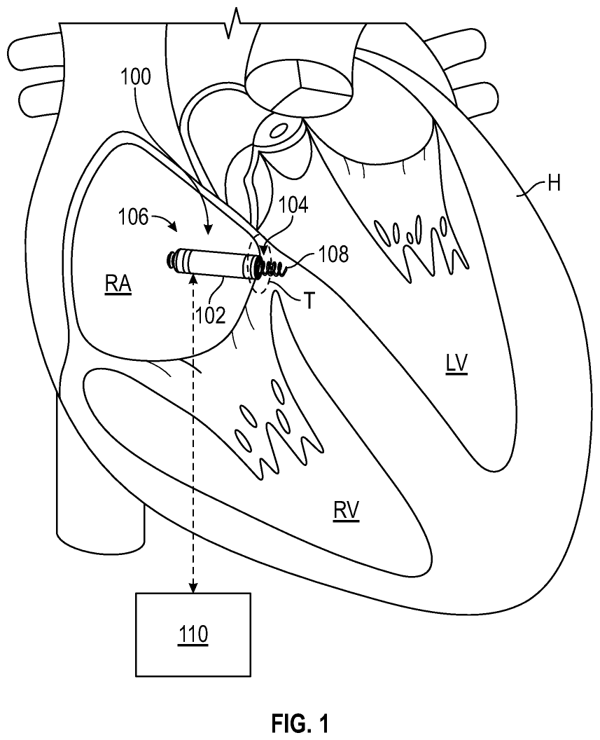

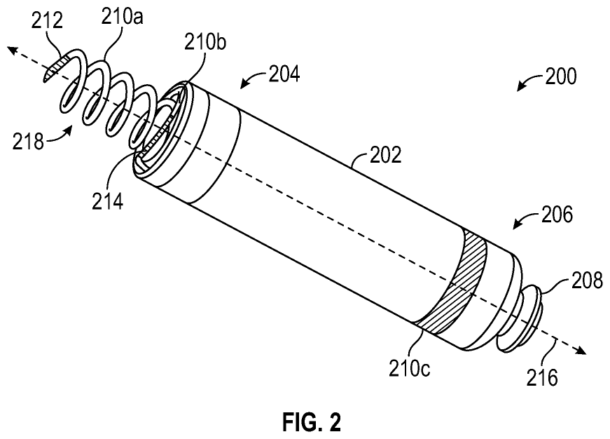

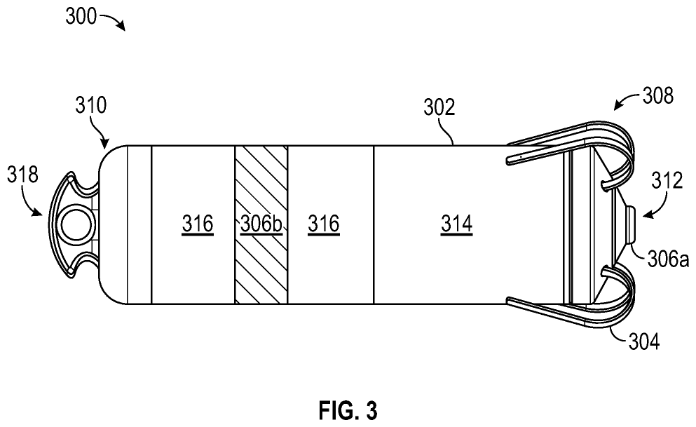

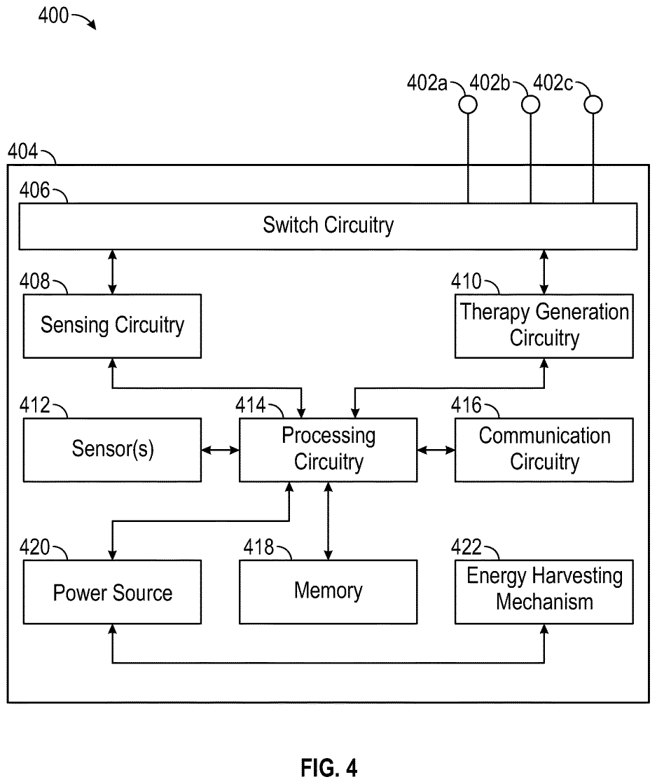

FIGS. 1-4 provide a general overview of implantable devices configured in accordance with embodiments of the present technology. Specifically, FIG. 1 illustrates a pacing device implanted in a patient's heart, FIG. 2 illustrates an example configuration for a pacing device, FIG. 3 illustrates another example configuration for a pacing device, and FIG. 4 illustrates electronic components that can be included in a pacing device. Any of the features of the embodiments of FIGS. 1-4 can be combined with each other and/or with any of the other embodiments described herein.

Referring first to FIG. 1, which illustrates a pacing device 100 implanted in the heart H of a patient, the device 100 is configured to monitor activity of the heart H and provide electrical stimulation (e.g., pacing signals) to the heart H. In some embodiments, the device 100 is a leadless intracardiac pacemaker configured to be implanted entirely within a heart chamber, such as entirely within the right atrium (RA), entirely within the right ventricle (RV), entirely within the left atrium (LA), or entirely within the left ventricle (LV). The device 100 can be implanted at any of a variety of locations to sense and/or deliver therapy to any chamber or chambers of the heart H. For example, as shown in FIG. 1, the device 100 can be a right atrial intracardiac pacemaker that is implanted in the RA of the patient's heart H in a target implant region T (e.g., the triangle of Koch). The target implant region T can lie between the bundle of His and the coronary sinus, and/or can be adjacent to the tricuspid valve. In other embodiments, the device 100 can instead be configured as a right ventricular intracardiac pacemaker that is implanted in the RV of the heart H, with the target implant region T lying along the endocardial wall at or near the apex of the RV.

The device 100 can include a housing 102 having a size and form factor suitable for transvenous delivery into the heart H via a catheter. In the illustrated embodiment, the housing 102 has an elongate shape extending from a distal portion 104 to a proximal portion 106. The housing 102 can have a generally cylindrical shape (e.g., pill-shaped or capsule-shaped), a generally prismatic shape (e.g., a rectangular prism), or any other suitable shape. The housing 102 can define an interior cavity that contains the electronic components of the device 100 (e.g., circuitry, power source, sensors).

The device 100 can include a fixation mechanism 108 to secure the device 100 to the tissue of the heart H. For example, the fixation mechanism 108 can include one or more fixation elements configured to penetrate into tissue, such as one or more tines, coils, barbs, etc. In the illustrated embodiment, the fixation mechanism 108 is coupled to and extends outwardly from the distal portion 104 of the housing 102. Accordingly, when the device 100 is implanted, the distal portion 104 can be positioned in contact with or in close proximity to the cardiac tissue, while the proximal portion 106 can be spaced apart from the cardiac tissue. In other embodiments, however, the fixation mechanism 108 can be located at a different portion of the device 100.

The device 100 also includes a plurality of electrodes configured to sense electrical activity of the heart H and/or deliver electrical therapy to the heart H. For example, the device 100 can include two, three, four, five, six, seven, eight, nine, ten, or more electrodes. Each electrode can be positioned at any suitable portion of the device 100, such as on or coupled to the housing 102 (e.g., the distal portion 104, the proximal portion 106, an intermediate location between the distal portion 104 and proximal portion 106), or on or coupled to the fixation mechanism 108. In some embodiments, the device 100 includes one or more electrodes (e.g., cathodes) that directly contact the cardiac tissue (e.g., of a single heart chamber or multiple heart chambers) to sense the activity thereof and/or deliver electrical therapy thereto. Such electrode(s) can be located at the distal portion 104 of the housing 102 and/or incorporated into the fixation mechanism 108, for example. The device 100 can also include at least one electrode (e.g., an anode and/or return electrode) that does not directly contact cardiac tissue. Such electrode(s) can be located at portions of the housing 102 that are spaced apart from cardiac tissue, such as the proximal portion 106. Optionally, a single electrode may serve as a cathode for certain operations, and may serve as an anode and/or return electrode for other operations.

In some embodiments, the device 100 is operably coupled to an external device 110 shown schematically) via bidirectional wireless communication, such as BLUETOOTH®, Wi-Fi, Medical Implant Communication Service (MICS), or other radiofrequency communication technique. The external device 110 can be a computing device or system that is located outside of the patient's body, and can be used in a healthcare setting (e.g., in a clinic, hospital or other medical facility), at the patient's home, or suitable combinations thereof. The external device 110 can be configured to control various operational parameters of the device 100, such as therapy parameters (e.g., pacing control parameters such as pacing interval), sensing parameters, power management parameters, etc. For instance, the external device 110 can transmit control signals to the device 100 to program one or more operational parameters of the device 100. Optionally, the external device 110 can display information relating to and/or received from the device 100, such as intracardiac electrogram (EGM) signals obtained by the device 100, motion sensor signals acquired by the device 100, operational parameters of the device 100, etc. In some embodiments, the external device 110 transmits information received from the device 100 to another computing device or system (e.g., a computer, laptop, workstation, mobile device, server, remote patient management system) for display, processing, and/or storage, using any suitable wired or wireless communication technique. The external device 110 can serve as a “programmer” that allows a physician, patient, or other individual to monitor and/or control the operations of the device 100.

Although FIG. 1 illustrates a single device 100, the present technology is also applicable to implantable systems including multiple devices 100 implanted at different locations in the heart H. For example, an implantable system can include a first device 100 in the RA and a second device 100 in the RV. In such embodiments, each device 100 can independently have any of the features described herein.

FIG. 2 is a perspective view of a pacing device 200 configured in accordance with embodiments of the present technology. The device 200 is configured to be implanted within a chamber of a heart of the patient to monitor activity of the heart and/or provide electrical therapy (e.g., pacing therapy) to the heart. The device 200 includes a housing 202 having a size and form factor that allows the device 200 to be entirely implanted within a single chamber of the patient's heart. In the illustrated embodiment, the housing 202 has an elongate shape (e.g., a generally cylindrical shape, a generally prismatic shape) extending between a distal end 204 and proximal end 206. The housing 202 can define a hermetically sealed internal cavity for housing the electronic components of the device 200. The housing 202 can also include an attachment mechanism 208 (e.g., at the proximal end 206) configured to temporarily engage with a delivery tool during implantation and/or extraction of the device 200.

The housing 202 can be formed partially or entirely from a conductive material, such as titanium or titanium alloy, stainless steel, MP35N (a non-magnetic nickel-cobalt-chromium-molybdenum alloy), a platinum alloy, or other biocompatible metal or metal alloy, or other suitable conductive material. Alternatively or in combination, the housing 202 can be formed partially or entirely from a nonconductive (e.g., insulative) material, such as ceramic, glass, sapphire, silicone, polyurethane, epoxy, acetyl co-polymer plastics, polyether ether ketone (PEEK), a liquid crystal polymer, other biocompatible polymer, or other suitable nonconductive material.

The device 200 can include a plurality of electrodes 210a-210c configured to sense electrical activity of the heart and/or deliver electrical stimulation to the heart. In the illustrated embodiment, for example, the device 200 includes a first electrode 210a and a second electrode 210b at or proximate to the distal end 204 of the housing 202, and a third electrode 210c on the housing 202. The first and second electrodes 210a, 210b can be configured as cathode electrodes that directly contact cardiac tissue, e.g., a distal end of the first electrode 210a can be configured to rest within a ventricular myocardium of the patient, and the second electrode 210b can be configured to contact an atrial endocardium of the patient. The third electrode 210c can be configured as an anode and/or return electrode that does not directly contact cardiac tissue.

As shown in FIG. 2, the first electrode 210a can be an elongate structure that extends from the distal end 204 of the housing 202 to penetrate through the wall tissue of a first heart chamber (e.g., the chamber in which the device 200 is implanted) into wall tissue of a second, different heart chamber. For example, in some embodiments, the device 200 is implanted in the RA with the distal end 204 oriented toward the LV (e.g., similar to the arrangement of the device 100 in FIG. 1), and the first electrode 210a extends through the wall tissue of the RA and into the wall tissue of the LV. In the illustrated embodiment, the first electrode 210a is configured as a coil (e.g., a helical and/or spiral coil), while in other embodiments, the first electrode 210a can have a different form factor (e.g., an elongate dart, barb, tine, or other tissue penetrating element). The first electrode 210a can include a proximal end that is coupled to the distal end 204 of the housing 202, and a free distal end that is not attached to the housing 202. The distal end of the first electrode 210a can have a conical, hemi-spherical, or slanted edge distal tip with a narrow tip diameter (e.g., less than 1 mm) for penetrating into and through tissue layers. In some embodiments, the distal end of the first electrode 210a can have a sharpened or angular tip, and/or sharpened or beveled edges, but the degree of sharpness can be constrained to avoid a cutting action that could lead to lateral displacement of the distal end of the first electrode 210a and undesired tissue trauma.

The second electrode 210b can be a structure that extends from the distal end 204 of the housing 202 to contact the wall tissue of the first heart chamber without penetrating the wall tissue. The second electrode 210b can be located proximal to the first electrode 210a. The second electrode 210b can be configured as a coil (e.g., a partial helical and/or spiral coil that does not form a full turn), loop, button, pad, or any other suitable form factor. The second electrode 210b can include a proximal end that is coupled to the distal end 204 of the housing 202, and a distal end that may or may not be coupled to the housing 202. In some embodiments, the second electrode 210b is configured to flexibly maintain contact with wall tissue of the heart chamber in which the device 200 is implanted, (e.g., the RA endocardium), despite variations in the tissue surface and/or in the distance between the distal end 204 of the housing 202 and the tissue surface, which may occur as the wall tissue moves during the cardiac cycle. Accordingly, the second electrode 210b can be flexible and/or have spring-like properties, e.g., the second electrode 210b can have a spring bias that urges at least a portion of the second electrode 210b away from the distal end 204 of the housing 202 and toward the wall tissue of the heart chamber to maintain consistent contact.

The first and second electrodes 210a, 210b can each be formed of an electrically conductive material, such as titanium, platinum, iridium, tantalum, or alloys thereof. The first electrode 210a can include one or more insulative coatings (e.g., parylene, polyurethane, silicone, epoxy) that reduce the electrically conductive surface area of the first electrode 210a to define a first electrically active region 212 (e.g., at or near the distal end of the first electrode 210a). The second electrode 210b can include one or more insulative coatings (e.g., parylene, polyurethane, silicone, epoxy) that reduce the electrically conductive surface area of the second electrode 210b to define a second electrically active region 214 (e.g., at an intermediate region between the proximal and distal ends of the second electrode 210b). This approach can increase the electrical impedance of the first and second electrodes 210a, 210b, and thereby reduce the current delivered during a pacing pulse, which can conserve the power used by the device 200. In some embodiments, the first and second electrodes 210a, 210b include an electrically conductive material coating (e.g., TiN) on the first and second electrically active regions 212, 214, respectively, to define the active regions. The first and second electrodes 210a, 210b can be made of the same materials, or can be made of different materials.

All, substantially all, or a portion of the housing 202 can serve as a third electrode 210c (e.g., an anode and/or return electrode) during pacing and/or sensing. In some embodiments, the third electrode 210c partially or fully circumscribes a portion of the housing 202 at or near the proximal end 206. Although FIG. 2 illustrates the third electrode 210c as a singular band, in other embodiments, the third electrode 210c can include multiple segments spaced a distance apart along a longitudinal axis 216 of the housing 202 and/or around a perimeter of the housing 202. Additionally, the third electrode 210c can also be located at other positions along the housing 202, e.g., located at or near the distal end 204 or at other positions along the longitudinal axis 216.

In embodiments where the housing 202 is formed from a conductive material, one or more portions of the housing 202 can be electrically insulated by a nonconductive material, such as a coating of parylene, polyurethane, silicone, epoxy or other biocompatible polymer, or other suitable material. For the portions of the housing 202 without the nonconductive material, one or more discrete areas of the housing 202 with conductive material can be exposed to define the third electrode 210c. In embodiments where the housing 202 is formed from a nonconductive material, a conductive material can be applied to one or more discrete areas of the housing 202 to form the third electrode 210c. Optionally, the third electrode 210c can be a discrete component (e.g., a ring electrode) that is coupled to the housing 202.

The electrodes 210a-210c can be used to sense electrical activity of one or more heart chambers and/or to deliver electrical stimulation to one or more heart chambers. For example, the first electrode 210a can be paired with the second electrode 210b or the third electrode 210c to for sensing ventricular signals and delivering ventricular pacing pulses. As another example, the second electrode 210b can be paired with the first electrode 210a or the third electrode 210c for sensing atrial signals and delivering pacing pulses to the atrial myocardium. In a further example, the third electrode 210c can be paired at different times with both the first electrode 210a and the second electrode 210b for either ventricular or atrial functionality, respectively. As yet another example, the first electrode 210a and the second electrode 210b can be paired with each other with different polarities for atrial and ventricular functionality.

In some embodiments, the second electrode 210b is configured as an atrial cathode electrode for delivering pacing pulses to the atrial tissue at a target implant region in combination with the third electrode 210c. The second electrode 210b and the third electrode 210c can also be used to sense atrial P-waves for use in controlling atrial pacing pulses (e.g., delivered in the absence of a sensed P-wave) and for controlling atrial-synchronized ventricular pacing pulses delivered using the first electrode 210a as a cathode and the third electrode 210c as the return anode. The configuration of the electrodes 210a-210c illustrated in FIG. 2 allows the device 200 to sense cardiac signals from and/or deliver cardiac pacing to one or more chambers of the heart. For example, the present technology can facilitate the delivery of A-V synchronous pacing using a single device 200 implanted within a single heart chamber (e.g., the RA).

The device 200 can include a fixation mechanism 218 configured to fix the device 200 to cardiac tissue at a target implant region (e.g., the triangle of Koch). In the illustrated embodiment, the first electrode 210a and/or second electrode 210b at the distal end 204 of the housing 202 can serve as the fixation mechanism 218. In other embodiments, the fixation mechanism 218 can be a different component than the first electrode 210a and/or the second electrode 210b, such one or more separate barbs, tines, coils, darts, etc.

FIG. 3 is a side view of another pacing device 300 configured in accordance with embodiments of the present technology. The device 300 is configured to be implanted within a chamber of a heart of a patient to monitor activity of the heart and/or to provide electrical therapy to the heart. In the embodiment shown in FIG. 3, the device 300 includes a housing 302, a plurality of fixation tines 304, a first electrode 306a, and a second electrode 306b.

The housing 302 can have a size and form factor that allows the device 300 to be entirely implanted within a chamber of a heart of a patient. For example, as shown in FIG. 3, the housing 302 has a generally cylindrical (e.g., pill-shaped or capsule-shaped), elongate form factor extending between a distal end 308 and a proximal end 310. The housing 302 contains electronic components of the device 300, and can be hermetically or near-hermetically sealed to prevent fluid ingress into the housing 302. The materials used to form the housing 302 can include any of the conductive and nonconductive materials described above with respect to FIG. 2.

The device 300 can include a fixation mechanism configured to fix the device 300 to cardiac tissue at a target implant region (e.g., the endocardial wall near the apex of the RV). In the illustrated embodiment, the device 300 includes a plurality of fixation tines 304 extending from the distal end 308 of the housing 302 and configured to engage with cardiac tissue to secure the housing 302 at a fixed position within the chamber of the heart. The fixation tines 304 can be configured to anchor the housing 302 to the cardiac tissue such that the device 300 moves along with the cardiac tissue during cardiac contractions. The device 300 can include any suitable number of fixation tines 304, such as one, two, three, four, five, or more fixation tines 304. The fixation tines 304 can be fabricated from any suitable material, such as a shape memory material (e.g., Nitinol). Alternatively or in combination, the device 300 can be fixed to cardiac tissue using other types of fixation mechanisms, such as, but not limited to, barbs, coils, darts, and the like.

Optionally, the device 300 can include an attachment mechanism configured to temporarily couple the device 300 to a delivery tool, e.g., for delivery and/or extraction of the device 300. In the illustrated embodiment, for example, the proximal end 310 includes a flange 318 that defines an opening. The flange 318 can be attached to a tether (e.g., by threading the tether through the opening) that extends through an elongate shaft (e.g., a catheter) to implant or extract the device 300.

In some embodiments, the device 300 is configured to sense electrical activity of the heart and/or deliver electrical stimulation to the heart via the first electrode 306a and second electrode 306b (collectively, “electrodes 306”). The first electrode 306a can serve as a cathode configured to electrically contact cardiac tissue and deliver pacing pulses thereto, and the second electrode 306b can serve as an anode and/or a return electrode. Optionally, the device 300 can be equipped with multiple cathode electrodes. Such multiple cathode electrodes can be configured to electrically contact and deliver pacing pulses to cardiac tissue of a single heart chamber, or cardiac tissue of multiple heart chambers. In some such embodiments, the multiple cathode electrodes are configured to electrically contact and deliver pacing pulses to cardiac tissue of different heart chambers. For example, one cathode electrode can be configured to electrically contact and deliver pacing pulses to atrial tissue, and another cathode electrode may be configured to electrically contact and deliver pacing pulses to ventricular tissue.

The electrodes 306 can be configured in many different ways. For example, one or both of the electrodes 306 can be discrete components that are mechanically coupled to the housing 302. As another example, one or both of the electrodes 306 can be defined by an outer portion of the housing 302 that is electrically conductive. The electrodes 306 can be electrically isolated from each other. In some embodiments, a portion of the housing 302 is covered by or formed from an insulative material to isolate the electrodes 306 from each other and/or to provide a desired size and shape for one or both of the electrodes 306. The electrodes 306 can be electrically coupled to at least some of the internal electronic components of the device 300 within the housing 302 (e.g., sensing circuitry, electrical stimulation circuitry, or both).

In the illustrated embodiment, the first electrode 306a is located at the distal end 308 of the housing 302. The first electrode 306a may be referred to as a tip electrode, and the fixation tines 304 can be configured to anchor the device 300 to cardiac tissue such that the first electrode 306a maintains contact with the cardiac tissue. In some examples, the housing 302 includes an end cap 312 at the distal end 308, and the end cap 312 includes a feedthrough assembly to electrically couple the first electrode 306a to the electronics within the housing 302, while electrically isolating the first electrode 306a from the remaining portions of the housing 302, e.g., including the second electrode 306b and/or other conductive portions of the housing 302.

The second electrode 306b can be located on the housing 302 away from (e.g., proximal to) the first electrode 306a. As shown in FIG. 3, the housing 302 includes a first portion 314 and a second portion 316, with the first portion 314 being located proximal to the end cap 312, and the second portion 316 being located proximal to the first portion 314. The second portion 316 can optionally define at least part of a power source case that houses a power source (e.g., a battery) of the pacing device 300. In some embodiments, the second electrode 306b is located on the second portion 316, while in other embodiments, the second electrode 306b is located on the first portion 314.

In some embodiments, the second electrode 306b is a conductive portion of the housing 302 (e.g., an annular portion of the housing 302 that is made partially or entirely from a conductive material). Additionally or alternatively, the second electrode 306b can be a conductive material that is coated onto the material of the housing 302, or a discrete component (e.g., a ring electrode) that is coupled to the housing 302. The remaining portions of the housing 302 can include or be coated with an insulative material so that the second electrode 306b is electrically isolated from the rest of the housing 302 and/or from the first electrodes 306a.

FIG. 4 is a schematic block diagram illustrating electronic components of a pacing device 400 configured in accordance with embodiments of the present technology. Any of the electronic components shown in FIG. 4 can be incorporated into any of the embodiments of implantable devices described herein, such as the device 100 of FIG. 1, the device 200 of FIG. 2, or the device 300 of FIG. 3.

As shown in FIG. 4, the device 400 includes a plurality of electrodes 402a-402c that are electrically coupled to components within a housing 404 of the device 400. Although the device 400 is illustrated and described herein as having three electrodes 402a-402c (e.g., similar to the device 200 of FIG. 2), in other embodiments, the device 400 can be modified to include a different number of electrodes, such as two electrodes (e.g., similar to the device 300 of FIG. 3) or any other suitable number of electrodes.

At least some of the electrodes 402a-402c can be configured to contact tissue of one or more heart chambers, as described elsewhere herein. For example, as discussed above with respect to FIG. 2, the first electrode 402a can be configured to electrically contact and deliver electrical signals to tissue of a first heart chamber (e.g., ventricular tissue), and the second electrode 402b can be configured to electrically contact and deliver electrical signals to tissue of a second, different heart chamber (e.g., atrial tissue). The third electrode 402c can be an anode and/or return electrode that does not electrically contact heart tissue. Optionally, either the first electrode 402a or the second electrode 402b can be omitted, or the device 400 can include additional electrodes that electrically contact and deliver electrical signals to tissue of a heart chamber (e.g., the first heart chamber, the second heart chamber, or another heart chamber).

The device 400 includes a plurality of electronic components within the housing 404, such as switch circuitry 406, sensing circuitry 408, therapy generation circuitry 410, one or more sensors 412, processing circuitry 414, communication circuitry 416, memory 418, and/or a power source 420. The various circuitry can be or include programmable or fixed function circuitry configured to perform the operations described herein. One or more of the components of the device 400 shown in FIG. 4 can be part of an electronics assembly. For example, one or more of the switch circuitry 406, sensing circuitry 408, therapy generation circuitry 410, sensor(s) 412, processing circuitry 414, communication circuitry 416, and/or memory 418 can be mounted on a circuit board of an electronics assembly of the device 400.

The switch circuitry 406 can include one or more switches (e.g., a switch matrix, switch arrays, or other collection of switches), multiplexers, transistors, and/or other electrical circuitry. The switch circuitry 406 can selectively couple one or more of the electrodes 402a-402c to other components of the device 400 (e.g., the sensing circuitry 408 and/or the therapy generation circuitry 410). The subset of the electrodes 402a-402c to be used can depend on the particular operation of the device 400 that is being performed, such as whether the device 400 is sensing or delivering therapy, the locations of the heart being monitored or treated, etc. In some embodiments, the processing circuitry 414 determines which subset of the electrodes 402a-402c should be used for a particular operation, and controls the switch circuitry 406 to selectively couple those electrodes to the appropriate components of the device 400.

The sensing circuitry 408 can monitor signals from at least one of electrodes 402a-402c to monitor electrical activity of the heart, impedance, and/or other electrical phenomena. Sensing can be performed to determine heart rates and/or heart rate variability; and/or to detect ventricular dyssynchrony, arrhythmias (e.g., tachyarrhythmias), and/or other electrical signals. The sensing circuitry 408 can include filters, amplifiers, analog-to-digital converters, and/or other circuitry configured to sense cardiac electrical signals via one or more of the electrodes 402a-402c.

In some embodiments, the switch circuitry 406 as controlled by the processing circuitry 414 selectively couples the sensing circuitry 408 to selected combinations of the electrodes 402a-402c, e.g., to selectively sense the electrical activity of one or more chambers of the heart. For example, the switch circuitry 406 can couple each of the first electrode 402a and the second electrode 402b (in combination with the third electrode 402c) to respective sensing channels provided by the sensing circuitry 408 to sense electrical signals from the cardiac tissues in electrical contact with the first electrode 402a (e.g., ventricular tissue) and the second electrodes 402b (e.g., atrial tissue), respectively. In some embodiments, the sensing circuitry 408 is configured to detect events, (e.g., depolarizations) within the cardiac electrical signals, and to provide indications thereof to the processing circuitry 414. In this manner, the processing circuitry 414 can determine the timing of atrial and/or ventricular depolarizations, and can control the delivery of cardiac pacing (e.g., AV synchronized cardiac pacing) based thereon.

The therapy generation circuitry 410 can generate electrical stimulation signals, such as cardiac pacing pulses. The therapy generation circuitry 410 can be electrically coupled to one or more of the electrodes 402a-402c to deliver pulses to a portion of cardiac muscle within the heart via one or more of the electrodes 402a-402c. In some embodiments, the therapy generation circuitry 410 delivers pacing stimulation in the form of electrical pulses. The therapy generation circuitry 410 can include charging circuitry, and one or more charge storage devices (e.g., capacitors). Optionally, the therapy generation circuitry 410 can include switches and/or other circuitry to control when the charge storage devices are discharged to the electrodes 402a-402c.

The switch circuitry 406 as controlled by the processing circuitry 414 can direct electrical stimulation signals from the therapy generation circuitry 410 to a selected combination of the electrodes 402a-402c having selected polarities, e.g., to selectively deliver pacing pulses to the RA, RV, LV, and/or the interventricular septum of the heart. For example, in order to pace one or both of the ventricles, the switch circuitry 406 can electrically couple the first electrode 402a (e.g., which contacts wall tissue of a ventricle or the intraventricular septum) to the therapy generation circuitry 410 as a cathode, and to one or both of the second electrode 402b or the third electrode 402c to the therapy generation circuitry 410 as an anode. As another example, in order to pace the RA, the switch circuitry 406 can couple the second electrode 402b (e.g., which contacts the RA endocardium) to the therapy generation circuitry 410 as a cathode, and to one or both of the first electrode 402a or the third electrode 402c to the therapy generation circuitry 410 as an anode.

The processing circuitry 414 can include one or more processors, such as a microprocessor, a controller, a digital signal processor (DSP), an application specific integrated circuit (ASIC), a field-programmable gate array (FPGA), or equivalent discrete or analog logic circuitry. In some embodiments, the processing circuitry 414 can include multiple components, such as any combination of one or more microprocessors, controllers, DSPs, ASICs, and/or FPGAs, as well as other discrete or integrated logic circuitry. The functions attributed to the processing circuitry 414 herein may be embodied as software, firmware, hardware, or any combination thereof.

The processing circuitry 414 can control the therapy generation circuitry 410 to deliver stimulation therapy to a patient's heart according to therapy parameters, which can be stored in the memory 418. For example, the processing circuitry 414 can control the therapy generation circuitry 410 to deliver electrical pulses with the amplitudes, pulse widths, rates, frequencies, and/or electrode polarities specified by the therapy parameters. In this manner, the therapy generation circuitry 410 can deliver pacing pulses to the heart via one or more of the electrodes 402a-402c. The device 400 can use any combination of the electrodes 402a-402c to deliver therapy and/or detect electrical signals from the patient.

The memory 418 (e.g., a data storage device or other non-transitory medium) can store computer-readable instructions that, when executed by the processing circuitry 414, cause the device 400 to perform the various operations described herein. The memory 418 can include any volatile, non-volatile, magnetic, optical, or electrical media, such as a random-access memory (RAM), read only memory (ROM), non-volatile RAM (NVRAM), electrically-erasable programmable ROM (EEPROM), flash memory, or any other digital or analog media.

The sensor(s) 412 can include one or more sensing elements that transduce patient physiological activity to an electrical signal to sense values of a respective patient parameter. Sensor(s) 412 can include one or more motion sensors, optical sensors, chemical sensors, temperature sensors, pressure sensors, and/or any other types of sensors. The sensor(s) 412 can output patient parameter values to the processing circuitry 414 that can be used as feedback to control sensing and/or delivery of therapy by the device 400.

For example, the sensor(s) 412 can include at least one motion sensor, such as one or more inertial measurement units (IMUs), accelerometers, gyroscopes, electrical or magnetic field sensors, and/or other devices capable of detecting motion and/or the position of the device 400. The motion of the device 400 detected by the motion sensor may be indicative of cardiac events (e.g., paced activation of the ventricles), blood flow through the heart, patient posture, patient activity, and/or noise. The processing circuitry 414 can control and/or monitor the motion data produced by the motion sensor to identify one or more features of the cardiac contraction within the signal (e.g., on a beat-by-beat basis or otherwise) to facilitate delivery of therapy (e.g., delivery of ventricular pacing pulses in an atrial-synchronized manner). Optionally, the processing circuitry 414 can use the motion data to detect a current activity level of the patient, which can be used for rate-responsive pacing of the patient's heart.

The communication circuitry 416 is configured to allow the device 400 to wirelessly communicate with another device, such as a device external to the patient's body (e.g., the external device 110 of FIG. 1) and/or another device under the control of the processing circuitry 414. For instance, the processing circuitry 414 can receive updates to operational parameters from the other device, and/or can provide collected data, (e.g., sensed heart activity and/or other patient parameters) to the other device via the communication circuitry 416. The communication circuitry 416 can use radiofrequency (RF) communication techniques (e.g., via an antenna) and/or any other suitable communication modality.

The power source 420 delivers operating power to various components of the device 400. The power source 420 can include one or more batteries, each of which can independently be rechargeable or non-rechargeable. Recharging of the power source 420 can be accomplished through proximal inductive interaction between an external charger and an inductive charging coil within the device 400. Alternatively or in combination, recharging of the power source 420 can be accomplished using an energy harvesting mechanism 422 of the device 400. Additional details of energy harvesting mechanisms suitable for use with the embodiments herein are provided in Section II below.

The components of the device 400 illustrated in FIG. 4 can be modified in many different ways. For example, any of the components shown in FIG. 4 can be combined with each other, e.g., the switch circuitry 406 can be incorporated into the sensing circuitry 408 and/or the therapy generation circuitry 410. Any of the components shown in FIG. 4 can be divided into smaller subcomponents. Some of the components in FIG. 4 are optional and may be omitted (e.g., the switch circuitry 406 and/or sensor(s) 412). The device 400 can also include additional components not shown in FIG. 4. For example, the device 400 can include power management circuitry coupled to the power source 420 to allow the processing circuitry 414 to monitor the status of the power source 420 (e.g., charge level, charging rate, net power into and/or out of the power source 420, remaining battery life).

The components of the device 400 shown in FIG. 4 represent functionality that can be included in any of the devices of the present technology. The components illustrated in FIG. 4 can include any discrete and/or integrated electronic circuit components that implement analog and/or digital circuits capable of producing the functions attributed to the components herein. For example, the components can include analog circuits, such as amplification circuits, filtering circuits, and/or other signal conditioning circuits. The components can also include digital circuits, such as combinational or sequential logic circuits, memory devices, and the like. The functions attributed to the components of FIG. 4 may be embodied as one or more processors, hardware, firmware, software, or any combination thereof. The depiction of different features as separate blocks in FIG. 4 is intended to highlight different functional aspects, and does not necessarily imply that such components must be realized by separate hardware or software components. Rather, functionality associated with one or more components may be performed by separate hardware or software components, or integrated within common or separate hardware or software components. For example, although illustrated as separate functional components in FIG. 4, some or all of the functionality attributed to the switch circuitry 406, sensing circuitry 408, therapy generation circuitry 410, sensor(s) 412, and/or communication circuitry 416 can alternatively or additionally be implemented by the processing circuitry 414, or vice-versa.

### II. Compact Energy Harvesting Mechanisms

In some embodiments, the present technology provides implantable devices that include an energy harvesting mechanism (also known as an “energy harvester” or “harvester”). The power capacity of a power source of an implantable device may be limited due to size constraints, such as if the device is implanted within a small space within the patient's body (e.g., within a single heart chamber) and/or to avoid the device interfering with normal physiological function, as well as safety considerations. To prolong the usable life of such implantable devices, an energy harvesting mechanism can be used to generate energy in situ to recharge the power source.

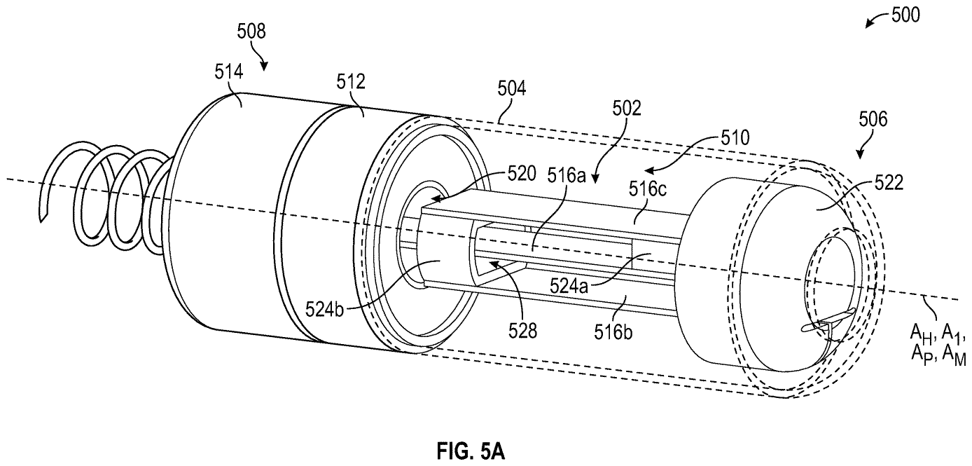

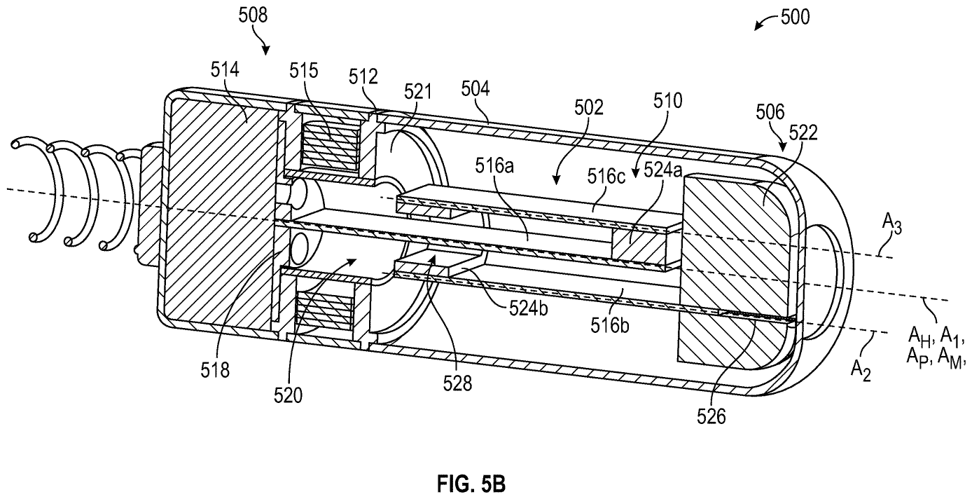

FIGS. 5A and 5B are perspective and cross-sectional views, respectively, of a device 500 including an energy harvesting mechanism 502, in accordance with embodiments of the present technology. Referring to FIGS. 5A and 5B together, the device 500 can be an implantable device, such as a pacing device configured to monitor activity of a patient's heart and provide electrical stimulation to the heart. In such embodiments, the device 500 can include any of the features of the devices described above in connection with FIGS. 1-4 (e.g., electrodes, fixation mechanism, circuitry, and/or other electronic components). In other embodiments, however, the device 500 can be a different type of implantable medical device.

The device 500 includes a housing 504 having a proximal end 506 and a distal end 508 (the housing 504 is depicted with broken lines in FIG. 5A for purposes of clarity). The housing 504 can have an elongate shape defining a central longitudinal axis A_{H} extending from the proximal end 506 to the distal end 508. The housing 504 includes an interior cavity 510 containing the energy harvesting mechanism 502 and other components of the device 500, such as a power assembly 512 and an electronics assembly 514 (shown schematically). When the device 500 is implanted in a patient's body, the energy harvesting mechanism 502 generates energy from physiological motion. For example, in some embodiments, the device 500 is configured to be implanted within a heart chamber of the patient and generates energy from cardiac motion (e.g., motion of the heart wall to which the device 500 is affixed) and/or blood flow through the heart chamber. The energy produced by the energy harvesting mechanism 502 can be used to charge the power assembly 512 that powers the operation of the device 500. The power assembly 512 can include one or more power sources 515, such as one or more accumulators (e.g., rechargeable batteries), that are electrically coupled to the energy harvesting mechanism 502 to store the energy produced by the energy harvesting mechanism 502.

The energy harvesting mechanism 502 includes a plurality of piezoelectric elements 516a-516c (collectively, “piezoelectric elements 516”) that convert mechanical energy into electrical energy via the piezoelectric effect. Although the illustrated embodiment shows three piezoelectric elements 516, in other embodiments, the device 500 can include a different number of piezoelectric elements 516, as described further below. Each piezoelectric element 516 can be made partially or entirely out of a piezoelectric material, such as a piezoelectric ceramic (e.g., lead zirconate titanate (PZT)), a single crystal piezoelectric material (e.g., lead magnesium niobate-lead titanate (PMN-PT)), a piezoelectric polymer (e.g., polyvinylidene difluoride (PVDF)), or a piezoelectric composite (e.g., a piezoelectric ceramic embedded in a polymer matrix, such as a macro fiber composite). In some embodiments, some or all of the piezoelectric elements 516 are piezoelectric bimorphs (e.g., piezoelectric bimorph beams) including two active layers of a piezoelectric material (e.g., an upper layer and a lower layer). Optionally, a piezoelectric bimorph can include a passive layer made out of a non-piezoelectric material (e.g., a polymer or a metal) that serves as a support for the active layers. In other embodiments, however, some or all of the piezoelectric elements 516 can be a piezoelectric unimorph having a single active layer.

The piezoelectric elements 516 can have any suitable geometry. Although the piezoelectric elements 516 are depicted as having a flattened, generally rectangular shape with a uniform width and thickness, in other embodiments, the piezoelectric elements 516 can have a different shape, such as a tapered shape with a varying width and/or thickness. In some embodiments, each piezoelectric element 516 has a flexible elongate body (e.g., a beam, plate, shaft, rod, fiber) extending from a respective first (e.g., distal) end portion to a respective second (e.g., proximal) end portion to define respective central longitudinal axes A₁, A₂, A₃ (FIG. 5B). The piezoelectric elements 516 can be arranged so that their respective central longitudinal axes A₁, A₂, A₃ are aligned with (e.g., parallel to or within 10°, 5°, 2°, or 1° of being parallel to) each other and/or to the central longitudinal axis A_{H} of the housing 504. In addition, the piezoelectric elements 516 can be arranged so that the central longitudinal axis A₁ of the first piezoelectric element 516a can overlap (e.g., intersect, be coaxial with) the central longitudinal axis A_{H} of the housing 504. Alternatively, the central longitudinal axis A₁ of the first piezoelectric element 516a can be spaced apart from the central longitudinal axis A_{H} of the housing 504. The central longitudinal axes A₂, A₃ of the second piezoelectric element 516b and/or third piezoelectric element 516c can be spaced apart from the central longitudinal axis A_{H} of the housing 504.

The first piezoelectric element 516a can be coupled to a fixation region within the housing 504, which can be part of the housing 504 or part of another component that is in a fixed spatial configuration relative to the housing 504. For example, as shown in FIG. 5B, the first piezoelectric element 516a can be mounted to an interior wall 518 (e.g., a bulkhead) within the housing 504. The interior wall 518 can be positioned between the electronics assembly 514 and the power assembly 512, e.g., proximal to the electronics assembly 514 and distal to the power assembly 512. Alternatively, the interior wall 518 can be at a different location, such as proximal to the power assembly 512 or distal to the electronics assembly 514. The first piezoelectric element 516a can be electrically coupled to the power source 515 of the power assembly 512 via electrical interconnections (e.g., wires) extending through and/or along the interior wall 518. In other embodiments, the first piezoelectric element 516a can instead be mounted to the power assembly 512, or to any other suitable part of the device 500.

In some embodiments, at least a portion of the first piezoelectric element 516a passes through the power assembly 512. As best seen in FIG. 5A, the power assembly 512 can be a hollow (e.g., tubular, annular) structure having a lumen 520 extending therethrough. For instance, the power assembly 512 can include a tubular outer housing 521 (e.g., a case or other enclosure) forming the lumen 520, and the power source 515 can be positioned within the outer housing 521. Although the power source 515 is depicted as also having a tubular shape with a respective lumen aligned with the lumen 520 of the power assembly 512, in other embodiments, the power source 515 can have a non-tubular shape and/or may not include a lumen. The first piezoelectric element 516a can be partially received within the lumen 520 of the power assembly 512. For example, at least part of the distal end portion of the first piezoelectric element 516a can be positioned within the lumen 520. In some embodiments, at least 5%, 10%, 15%, 20%, 25%, 30%, 35%, 40%, 45%, 50%, of the total length of the first piezoelectric element 516a is received within the lumen 520, and/or no more than 50%, 45%, 40%, 35%, 30%, 25%, 20%, 15%, 10%, 5% of the total length of the first piezoelectric element 516a is received within the lumen 520. The power assembly 512 can have a central longitudinal axis A_{P} that extends through the lumen 520. The central longitudinal axis A_{P} of the power assembly 512 can be aligned with (e.g., parallel to) the central longitudinal axis A_{H} of the housing 504. The central longitudinal axis A_{P} of the power assembly 512 can overlap (e.g., intersect, be coaxial with) the central longitudinal axis A_{H} of the housing 504, or can be spaced apart from the central longitudinal axis A_{H} of the housing 504.

The central longitudinal axis A₁ of the first piezoelectric element 516a can be aligned with (e.g., parallel to) the central longitudinal axis A_{P} of the power assembly 512. As shown in FIG. 5B, the central longitudinal axis A₁ of the first piezoelectric element 516a can overlap (e.g., intersect, be coaxial with) the central longitudinal axis A_{P} of the power assembly 512. This configuration can be advantageous for reducing the overall size of the device 500 while maintaining sufficient space within the interior cavity 510 to allow for movement of the harvester mass 522 and piezoelectric element 516a. In other embodiments, however, the power assembly 512 can have a different shape (e.g., a solid shape without any lumen) and/or can be positioned differently with respect to the energy harvesting mechanism 502 (e.g., the first piezoelectric element 516a can be entirely outside of the power assembly 512, the central longitudinal axis A₁ of the first piezoelectric element 516a can be spaced apart from the central longitudinal axis A_{P} of the power assembly 512).

The second piezoelectric element 516b can be coupled to a harvester mass 522 (also known as a “proof mass” or “inertial mass”), which can be movable within the interior cavity 510 of the housing 504, as described in greater detail below. The harvester mass 522 can include an opening 526 (e.g., slot, recess, groove, cavity) and a portion of the second piezoelectric element 516b (e.g., the proximal end portion) can be received within the opening 526. Alternatively or in combination, the harvester mass 522 can be coupled to the second piezoelectric element 516b using bonding, adhesives, fasteners, etc. The harvester mass 522 can include a central axis A_{M}, which can pass through the center of mass of the harvester mass 522 or can pass through the harvester mass 522 in a location other than the center of mass. In the illustrated embodiment, the second piezoelectric element 516b is coupled to the harvester mass 522 at a location spaced apart from the central axis A_{M}, such that the second longitudinal axis A₂ is offset from the central axis A_{M}. Alternatively, the second piezoelectric element 516b can be coupled to the harvester mass 522 at a location along the central axis A_{M} such that the second longitudinal axis A₂ overlaps (e.g., intersects, is coaxial with) the central axis A_{M}.

The third piezoelectric element 516c can be coupled in series between the first piezoelectric element 516a and the second piezoelectric element 516b, with the first piezoelectric element 516a being coupled to the third piezoelectric element 516c, and the third piezoelectric element 516c being coupled to the second piezoelectric element 516b. In some embodiments, the piezoelectric elements 516 are coupled to each other in an end-to-end, folded configuration (e.g., a fan-folded, zig-zag, or serpentine configuration). For example, the distal end portion of the first piezoelectric element 516a can be affixed relative to the housing 504 (e.g., to the interior wall 518). The proximal end portion of the first piezoelectric element 516a can be coupled to the proximal end portion of the third piezoelectric element 516c (e.g., via a first joint 524a). The distal end portion of the third piezoelectric element 516c can be coupled to the distal end portion of the second piezoelectric element 516b (e.g., via a second joint 524b). The proximal end portion of the second piezoelectric element 516b can be coupled to the harvester mass 522.

In some embodiments, the second piezoelectric element 516b and third piezoelectric element 516c are positioned at different sides of the first piezoelectric element 516a. For example, as shown in FIGS. 5A and 5B, the second piezoelectric element 516b and third piezoelectric element 516c can be disposed at opposite sides of the first piezoelectric element 516a (e.g., upper and lower sides, lateral sides). Alternatively, the second and third piezoelectric elements 516b, 516c can be disposed at adjacent sides of the first piezoelectric element 516a (e.g., upper side and lateral side, lower side and lateral side). The central longitudinal axis A₂ of the second piezoelectric element 516b and/or the central longitudinal axis A₃ of the third piezoelectric element 516c can be spaced apart from the central longitudinal axis A_{H} of the housing 504 and/or from the central longitudinal axis A_{P} of the power assembly 512.

As shown in FIGS. 5A and 5B, the second and/or third piezoelectric elements 516b, 516c can be longitudinally offset from the first piezoelectric element 516a, such that the distal end portions of the second and/or third piezoelectric elements 516b, 516c are located proximal to the distal end portion of the first piezoelectric element 516a. Accordingly, the second and/or third piezoelectric elements 516b, 516c can be proximal to the power assembly 512. In some embodiments, the second and/or third piezoelectric elements 516b, 516c are entirely outside of the lumen 520 of the power assembly 512. In other embodiments, however, the second and/or third piezoelectric elements 516b, 516c can be at least partially within the lumen 520 of the power assembly 512 (e.g., the distal end portions of the second and/or third piezoelectric elements 516b, 516c can be within the lumen 520).

The joints 524a, 524b can be blocks, strips, plates, brackets, wires, interposers, etc., that mechanically couple the respective pairs of piezoelectric elements 516 to each other. The joints 524a, 524b can be sufficiently rigid to maintain the piezoelectric elements 516 in the desired spatial arrangement relative to each other, and/or to reduce excessive flexing of the joints 524a, 524b that dissipates mechanical energy. Optionally, the joints 524a, 524b can have some degree of flexibility to provide an elastic rebound effect that dynamically amplifies the deflection of the piezoelectric elements 516 in response to a motion input.

For example, the first joint 524a can be a solid, continuous component (e.g., a block or a plate) interposed between the first piezoelectric element 516a and the third piezoelectric element 516c. In some embodiments, the first joint 524a includes a lower surface coupled to the upper surface of the first piezoelectric element 516a, and an upper surface coupled to the lower surface of the third piezoelectric element 516c. Although the first joint 524a is depicted as having a rectangular cross-sectional shape, in other embodiments, the first joint 524a can have a different cross-sectional shape, such as square, trapezoidal, etc.

The second joint 524b can be a hollow component that extends partially or entirely around a portion of the first piezoelectric element 516a. In some embodiments, the second joint 524b is an annular structure (e.g., a tube, ring, band, loop) or a portion thereof (e.g., half a tube, half a ring) that includes a recess 528 (e.g., a lumen, opening, cavity, slot) receiving the portion of the first piezoelectric element 516a. The second joint 524b can include an upper surface coupled to the lower surface of the third piezoelectric element 516c, and a lower surface coupled to the upper surface of the second piezoelectric element 516b. Additionally or alternatively, the second joint 524b can couple to the upper surface of the third piezoelectric element 516c and the lower surface of the second piezoelectric element 516b. Although the second joint 524b is depicted as having a stadium cross-sectional shape, in other embodiments, the second joint 524b can have a different cross-sectional shape, such as square, rectangular, trapezoidal, etc.

In some embodiments, the joints 524a, 524b include electrically conductive materials (e.g., metals, conductive polymers) to electrically couple the piezoelectric elements 516 to each other. Accordingly, current produced by the second piezoelectric element 516b and the third piezoelectric element 516c can be transmitted to the power assembly 512 via the joints 524a, 524b. For example, the joints 524a, 524b can be made entirely out of a metal (e.g., brass, copper), and can be attached to the corresponding piezoelectric elements 516 via soldering, brazing, conductive adhesives, etc. As another example, the joints 524a, 524b can be made out of a combination of an insulative material (e.g., polyimide, polycrystalline ceramic such as alumina) and a conductive material (e.g., conductive traces, fine wires) using techniques such as molding and hot isostatic processing. In other embodiments, however, the joints 524a, 524b may not include any electrically conductive materials and/or may be used to mechanically couple the piezoelectric elements 516 without electrically coupling the piezoelectric elements 516. In such embodiments, each piezoelectric element 516 can be electrically coupled to the power assembly 512 via a respective wire, trace, or other electrical connector. Moreover, one or both of the joints 524a, 524b can be omitted, and the piezoelectric elements 516 can instead be directly (e.g., mechanically and/or electrically) connected to each other (e.g., via welding, adhesives, fasteners) or integrally formed with each other.

The configuration of the piezoelectric elements 516 described herein can decrease the overall resonant frequency of the energy harvesting mechanism 502 to a range suitable for efficient harvesting from physiological motion, while also reducing the spatial footprint of the energy harvesting mechanism 502. For example, the resonant frequency of the energy harvesting mechanism 502 can be within a range from 1 Hz to 50 Hz, 1 Hz to 30 Hz, 1 Hz to 20 Hz, 1 Hz to 10 Hz, 5 Hz to 10 Hz, 5 Hz to 15 Hz, 10 Hz to 20 Hz, 10 Hz to 15 Hz, 10 Hz to 30 Hz, 15 Hz to 20 Hz, 15 Hz to 25 Hz, 20 Hz to 30 Hz, 20 Hz to 25 Hz, or 25 Hz to 30 Hz. The length of the energy harvesting mechanism 502 (excluding the harvester mass 522) can be less than or equal to 30 mm, 25 mm, 20 mm, or 15 mm; and/or within a range from 10 mm to 30 mm, 10 mm to 20 mm, 14 mm to 18 mm, or 15 mm to 2mm. The maximum cross-sectional dimension of the energy harvesting mechanism 502 (e.g., maximum width and/or diameter measured orthogonal to the central longitudinal axis A_{H}) (excluding the harvester mass 522) can be less than or equal to within 15 mm, 12 mm, 10 mm, or 8 mm; and/or within a range from 5 mm to 15 mm, or 5 mm to 10 mm.

When the device 500 is subjected to external forces from physiological motion, the inertia of the harvester mass 522 can cause deflection of the proximal end portion of the second piezoelectric element 516b. The forces can be transmitted to the third piezoelectric element 516c and the first piezoelectric element 516a by virtue of the interconnections between these components. Accordingly, some or all of the piezoelectric elements 516 can be elastically deformed relative to the housing 504 and to the fixed distal end portion of the first piezoelectric element 516a. For example, some or all of the piezoelectric elements 516 can be deformed from a resting, straightened configuration (shown in FIGS. 5A and 5B) to a bent configuration. The resulting mechanical strain in the piezoelectric element(s) 516 can produce an electrical current that can be used to charge the power assembly 512.

Optionally, the device 500 can include power conditioning circuitry (not shown) electrically coupled to and interposed between the energy harvesting mechanism 502 and the power assembly 512. The power conditioning circuitry can be configured to perform operations such as rectification, filtering, voltage regulation, etc., of the electrical signal produced by the energy harvesting mechanism 502, before transmission to the power assembly 512. In some embodiments, the power conditioning circuitry is part of the power assembly 512 and/or is positioned within the outer housing 521 of the power assembly 512.

The power assembly 512 is electrically coupled to the electronics assembly 514 to power the operation thereof. The electronics assembly 514 can include the electronic components of the device 500, such as any of the components described above with respect to FIG. 4 (e.g., switch circuitry 406, sensing circuitry 408, therapy generation circuitry 410, sensors 412 processing circuitry 414, communication circuitry 416, and/or memory 418).

Optionally, the power assembly 512 and/or the electronics assembly 514 can include components (e.g., processing circuitry 414, capacitors, and/or other circuitry) that perform power management functions, such as monitoring the status of the power assembly 512 (e.g., the charge level of the power source 515; whether the charge level is increasing, decreasing, or constant; the net current and/or power into the power source 515) and/or monitoring the power output of the energy harvesting mechanism 502 (e.g., amount of current and/or power produced by the energy harvesting mechanism 502), power consumption of the electronics assembly 514, etc.

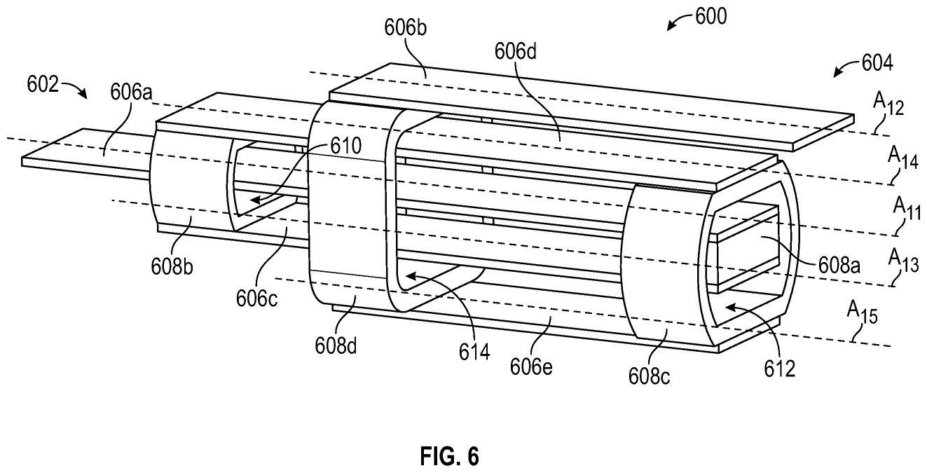

FIG. 6 is a perspective view of an energy harvesting mechanism 600, in accordance with embodiments of the present technology. The energy harvesting mechanism 600 can be incorporated into any of the devices described herein. For example, the energy harvesting mechanism 600 can be substituted for the energy harvesting mechanism 502 of FIGS. 5A and 5B. In some embodiments, the energy harvesting mechanism 600 generates energy from physiological motion (e.g., cardiac motion). The energy produced by the energy harvesting mechanism 600 can be used to charge a power source (e.g., the power source 515 of the device 500).

The energy harvesting mechanism 600 includes a distal end 602, a proximal end 604, and five piezoelectric elements 606a-606e (collectively, “piezoelectric elements 606”) that convert mechanical energy into electrical energy via the piezoelectric effect. The piezoelectric elements 606 can be identical or generally similar to the piezoelectric elements 516 of FIGS. 5A and 5B. For example, each piezoelectric element 606 can be made partially or entirely out of a piezoelectric material, such as any of the materials described herein. Some or all of the piezoelectric elements 606 can be piezoelectric bimorphs, or some or all of the piezoelectric elements 606 can be piezoelectric unimorphs.

The piezoelectric elements 606 can have any suitable geometry. Although the piezoelectric elements 606 are each depicted as having a flattened, generally rectangular shape with a uniform width and thickness, in other embodiments, the piezoelectric elements 606 can have a different shape, such as a tapered shape with a varying width and/or thickness. In some embodiments, each piezoelectric element 606 has a flexible elongate body (e.g., a beam, plate, shaft, rod, fiber) extending from a respective first (e.g., distal) end portion to a respective second (e.g., proximal) end portion to define respective central longitudinal axes A₁₁-A₁₅. The piezoelectric elements 606 can be arranged so that their respective central longitudinal axes A₁₁-A₁₅ are aligned with (e.g., parallel to or within 10°, 5°, 2°, or 1° of being parallel to) each other and/or to the central longitudinal axis of a housing containing the energy harvesting mechanism 600 (e.g., the central longitudinal axis A_{H} of the housing 504). In addition, the piezoelectric elements 606 can be arranged so that the central longitudinal axis A₁₁ of the first piezoelectric element 606a can overlap (e.g., intersect, be coaxial with) the central longitudinal axis of the housing. Alternatively, the central longitudinal axis A₁₁ of the first piezoelectric element 606a can be spaced apart from the central longitudinal axis of the housing. The central longitudinal axes A₁₂-A₁₅ of the second, third, fourth, and/or fifth piezoelectric elements 606b-606e can be spaced apart from the central longitudinal axis of the housing.

The first piezoelectric element 606a can be generally similar to the first piezoelectric element 516a of FIGS. 5A and 5B. For example, the distal end portion of the first piezoelectric element 606a can be coupled to a fixation region within the housing (not shown in FIG. 6), which can be part of the housing or part of another component that is in a fixed spatial configuration relative to the housing. The first piezoelectric element 606a can be mounted to an interior wall within the housing (e.g., the interior wall 518 within the housing 504). The first piezoelectric element 606a can be electrically coupled to a power assembly (e.g., power assembly 512) via electrical connections (e.g., wires) extending through and/or along the interior wall.

In some embodiments, the power assembly is a hollow structure having a lumen (e.g., the lumen 520 of power assembly 512), and at least a portion of the first piezoelectric element 606a passes through and is received within the lumen. For example, at least part of the distal end portion of the first piezoelectric element 606a can be positioned within the lumen. In some embodiments, at least 5%, 10%, 15%, 20%, 25%, 30%, 35%, 40%, 45%, 50%, of the total length of the first piezoelectric element 606a is received within the lumen, and/or no more than 50%, 45%, 40%, 35%, 30%, 25%, 20%, 15%, 10%, 5% of the total length of the first piezoelectric element 606a is received within the lumen. The central longitudinal axis A₁₁ of the first piezoelectric element 606a can be aligned with (e.g., parallel to) the central longitudinal axis of the power assembly (e.g., the central longitudinal axis A_{P} of the power assembly 512). Optionally, the central longitudinal axis A₁₁ of the first piezoelectric element 606a can overlap (e.g., intersect, be coaxial with) the central longitudinal axis of the power assembly. In other embodiments, however, the power assembly can have a different shape (e.g., a solid shape without any lumen), and/or can be positioned differently with respect to the energy harvesting mechanism 600 (e.g., the first piezoelectric element 606a can be entirely outside of the power assembly, the central longitudinal axis A₁₁ of the first piezoelectric element 606a can be spaced apart from the central longitudinal axis of the power assembly).

The second piezoelectric element 606b can be generally similar to the second piezoelectric element 516b of FIGS. 5A and 5B. For example, the proximal end portion of the second piezoelectric element 606b can be coupled to a harvester mass (e.g., the harvester mass 522 of the energy harvesting mechanism 502—not shown in FIG. 6). In some embodiments, the proximal end portion fits within an opening in the harvester mass (e.g., the opening 526 of the harvester mass 522) and/or is coupled to the harvester mass using bonding, adhesives, fasteners, etc. The second piezoelectric element 606b can be coupled to the harvester mass at a location spaced apart from the central axis of the harvester mass (e.g., the central axis A_{M} of the harvester mass 522) or along the central axis of the harvester mass.

The piezoelectric elements 606c-606e can be coupled in series between the first piezoelectric element 606a and the second piezoelectric element 606b, with the first piezoelectric element 606a being coupled to the third piezoelectric element 606c, the third piezoelectric element 606c being coupled to the fourth piezoelectric element 606d, the fourth piezoelectric element 606d being coupled to the fifth piezoelectric element 606e, and the fifth piezoelectric element 606e being coupled to the second piezoelectric element 606b. In some embodiments, the piezoelectric elements 606 are coupled to each other in an end-to-end, folded configuration (e.g., a fan-folded, zig-zag, or serpentine configuration). For example, the distal end portion of the first piezoelectric element 606a can be affixed relative to the housing (e.g., to the interior wall). The proximal end portion of the first piezoelectric element 606a can be coupled to the proximal end portion of the third piezoelectric element 606c (e.g., via a first joint 608a). The distal end portion of the third piezoelectric element 606c can be coupled to the distal end portion of the fourth piezoelectric element 606d (e.g., via a second joint 608b). The proximal end portion of the fourth piezoelectric element 606d can be coupled to the proximal end portion of the fifth piezoelectric element 606e (e.g., via a third joint 608c). The distal end portion of the fifth piezoelectric element 606e can be coupled to the distal end portion of the second piezoelectric element 606b (e.g., via a fourth joint 608d). The proximal end portion of the second piezoelectric element 606b can be coupled to the harvester mass.

In some embodiments, at least some of the piezoelectric elements 606b-606e are positioned at different sides of the first piezoelectric element 606a (e.g., upper and lower sides, lateral sides). For instance, as shown in FIG. 6, the second piezoelectric element 606b and fourth piezoelectric element 606d can be positioned at a first side of the first piezoelectric element 606a (e.g., an upper side), with the fourth piezoelectric element 606d interposed between the first and second piezoelectric elements 606a, 606b. The third piezoelectric element 606c and the fifth piezoelectric element 606e can be positioned at a second, opposite side of the first piezoelectric element 606a (e.g., a lower side), with the third piezoelectric element 606c interposed between the first and fifth piezoelectric elements 606a, 606e. Additionally, the central longitudinal axes A₁₂-A₁₅ of some or all of the piezoelectric elements 606b-606e can be spaced apart from the central longitudinal axis of the housing and/or the central longitudinal axis of the power assembly. In other embodiments, however, some or all of the piezoelectric elements 606b-606e can be arranged differently, as described further below.

As shown in FIG. 6, some or all of the piezoelectric elements 606b-606e can be longitudinally offset from the first piezoelectric element 606a, such that some or all of the distal end portions of the piezoelectric elements 606b-606e are located proximal to the distal end portion of the first piezoelectric element 606a. Additionally, the distal end portion of the second piezoelectric element 606b can be proximal to the distal end portion of the fourth piezoelectric element 606d, and/or the distal end portion of the fifth piezoelectric element 606e can be proximal to the distal end portion of the third piezoelectric element 606c. Some or all of the piezoelectric elements 606b-606e can be proximal to the power assembly and/or located entirely outside of the lumen of the power assembly. In other embodiments, some or all of the piezoelectric elements 606b-606e can be at least partially within the lumen of the power assembly (e.g., the distal end portions of some or all of the piezoelectric elements 606b-606e can be within the lumen).

The joints 608a-608d can be generally similar to the joints 524a, 524b of FIGS. 5A and 5B. For example, the joints 608a-608d can be blocks, strips, plates, brackets, wires, interposers, etc., that mechanically couple the respective pairs of piezoelectric elements 606 to each other. The joints can be rigid components (e.g., to maintain the piezoelectric elements 606 in the desired spatial arrangement relative to each other, and/or to reduce excessive flexing), or can have some degree of flexibility (e.g., to provide an elastic rebound effect that dynamically amplifies the deflection of the piezoelectric elements 606 in response to a motion input). In some embodiments, some or all of the joints 608a-608d include electrically conductive materials as described herein to electrically couple the piezoelectric elements 606 to each other. Accordingly, current produced by the piezoelectric elements 606b-606e can be transmitted to the power assembly via the joints 608a-608d. In other embodiments, however, the joints 608a-608d may not include any electrically conductive materials and/or may be used to mechanically couple the piezoelectric elements 606 without electrically coupling the piezoelectric elements 606. In such embodiments, each piezoelectric element 606 can be electrically coupled to the power assembly via a respective electrical connector. Moreover, some or all of the joints 608a-608d can be omitted, and the corresponding piezoelectric elements 606 can instead be directly (e.g., mechanically and/or electrically) connected to each other or integrally formed with each other.

The first joint 608a can be a solid, continuous component (e.g., a block or a plate) interposed between the first piezoelectric element 606a and the third piezoelectric element 606c. In some embodiments, the first joint 608a includes an upper surface coupled to the lower surface of the first piezoelectric element 606a, and a lower surface coupled to the upper surface of the third piezoelectric element 606c. Although the first joint 608a is depicted as having a rectangular cross-sectional shape, in other embodiments, the first joint 608a can have a different cross-sectional shape, such as square, trapezoidal, etc.

The second joint 608b can be a hollow component that extends partially or entirely around a portion of the first piezoelectric element 606a. In some embodiments, the second joint 608b is an annular structure (e.g., a tube, ring, band, loop) or a portion thereof (e.g., half a tube, half a ring) that includes a recess 610 (e.g., a lumen, opening, cavity, slot) receiving the portion of the first piezoelectric element 606a. The second joint 608b can include a lower surface coupled to the upper surface of the third piezoelectric element 606c, and an upper surface coupled to the lower surface of the fourth piezoelectric element 606d. Additionally or alternatively, the second joint 608b can couple to the upper surface of the fourth piezoelectric element 606d and the lower surface of the third piezoelectric element 606c. Although the second joint 608b is depicted as having a stadium cross-sectional shape, in other embodiments, the second joint 608b can have a different cross-sectional shape, such as square, rectangular, trapezoidal, etc.

The third joint 608c can be a hollow component (e.g., an annular structure or portion thereof) that extends partially or entirely around a portion of the first piezoelectric element 606a, third piezoelectric element 606c, and/or the first joint 608a. The third joint 608c can include a recess 612 (e.g., a lumen, opening, cavity, slot) that receives the portions of the first piezoelectric element 606a, third piezoelectric element 606c, and/or the first joint 608a. The third joint 608c can include a lower surface coupled to the upper surface of the fifth piezoelectric element 606e, and an upper surface coupled to the lower surface of the fourth piezoelectric element 606d. Additionally or alternatively, the third joint 608c can couple to the upper surface of the fourth piezoelectric element 606d and the lower surface of the fifth piezoelectric element 606e. Although the third joint 608c is depicted as having a stadium cross-sectional shape, in other embodiments, the third joint 608c can have a different cross-sectional shape, such as square, rectangular, trapezoidal, etc.

The fourth joint 608d can be a hollow component (e.g., an annular structure or portion thereof) that extends partially or entirely around a portion of the first, third, and/or fourth piezoelectric elements 606a, 606c, 606d. The fourth joint 608d can include a recess 614 (e.g., a lumen, opening, cavity, slot) that receives the portions of the first, third, and/or fourth piezoelectric elements 606a, 606c, 606d. The fourth joint 608d can include a lower surface coupled to the upper surface of the fifth piezoelectric element 606e, and an upper surface coupled to the lower surface of the second piezoelectric element 606b. Additionally or alternatively, the fourth joint 608d can couple to the upper surface of the second piezoelectric element 606b and the lower surface of the fifth piezoelectric element 606e. Although the fourth joint 608d is depicted as having a square cross-sectional shape, in other embodiments, the fourth joint 608d can have a different cross-sectional shape, such as stadium, rectangular, trapezoidal, etc.

Some or all of the joints 608a-608d can be longitudinally offset from each other. For instance, in the illustrated embodiment, the first joint 608a and third joint 608c are located near the proximal end 604 of the energy harvesting mechanism 600, the second joint 608b is located near the distal end 602 of the energy harvesting mechanism 600, and the fourth joint 608d is located at an intermediate position between the proximal end 604 and the distal end 602. Additionally, some or all of the joints 608a-608d can have different shapes and/or sizes. In some embodiments, the first joint 608a has a smaller cross-sectional dimension (e.g., height, width) than the second joint 608b, which has a smaller cross-sectional dimension than the third joint 608c, which has a smaller cross-sectional dimension than the fourth joint 608d. In other embodiments, however, the locations and/or sizes of any of the joints 608a-608d can be varied as desired (e.g., depending on the arrangement of the piezoelectric elements 606), as discussed further below.

The configuration of the piezoelectric elements 606 described herein can decrease the overall resonant frequency of the energy harvesting mechanism 600 to a range suitable for efficient harvesting from physiological motion, while also reducing the spatial footprint of the energy harvesting mechanism 600. For example, the resonant frequency of the energy harvesting mechanism 600, length of the energy harvesting mechanism 600 (excluding the harvester mass), and maximum cross-sectional dimension of the energy harvesting mechanism 600 can be identical or similar to the corresponding parameters of the energy harvesting mechanism 502 of FIGS. 5A and 5B.

When the energy harvesting mechanism 600 is subjected to external forces from physiological motion, the inertia of the harvester mass can cause deflection of the proximal end portion of the second piezoelectric element 606b. The forces can be transmitted to the other piezoelectric elements 606 by virtue of the interconnections between these components (e.g., joints 608a-608d). Accordingly, some or all of the piezoelectric elements 606 can be elastically deformed relative to the housing and to the fixed distal end portion of the first piezoelectric element 606a. For example, some or all of the piezoelectric elements 606 can be deformed from a resting, straightened configuration (shown in FIG. 6) to a bent configuration. The resulting mechanical strain in the piezoelectric element(s) 606 can produce an electrical current that can be used to charge the power source.

The configuration of the energy harvesting mechanism 600 of FIG. 6 can be modified in many ways. For example, the piezoelectric elements 606b-606e can be disposed on any combination of the same, adjacent, and/or opposite sides of the first piezoelectric element 606a. In some embodiments, the third piezoelectric element 606c and fourth piezoelectric element 606d are positioned at a first side (e.g., a lower side) of the first piezoelectric element 606a, with the third piezoelectric element 606c interposed between the first and fourth piezoelectric elements 606a, 606d. The second and fifth piezoelectric elements 606b, 606e can be positioned at a second, opposite side (e.g., an upper side) of the first piezoelectric element 606a, with the fifth piezoelectric element 606e interposed between the first and second piezoelectric elements 606a, 606b. As another example, the third piezoelectric element 606c can be disposed at a lower side of the first piezoelectric element 606a, the fourth piezoelectric element 606d can be disposed at one lateral side of the first piezoelectric element 606a, the fifth piezoelectric element 606e can be disposed at an upper side of the first piezoelectric element 606a, and the second piezoelectric element 606b can be disposed at the other lateral side of the first piezoelectric element 606a. Additionally, the locations and geometries of some or all of the joints 608a-608d can be modified to accommodate the different arrangements of the piezoelectric elements 606b-606e. For example, some or all of the joints 608b-608d can instead be solid components without recesses, some or all of the piezoelectric elements 606b-606e can instead be coupled to the lateral surfaces of the corresponding joints rather than to the upper and lower surfaces, etc.

Although the embodiments of FIGS. 5A-6 illustrate energy harvesting mechanisms with three and five piezoelectric elements, respectively, the techniques described herein can be applied to energy harvesting mechanisms with any suitable number of piezoelectric elements, such as two, three, four, five, six, seven, eight, nine, 10, 15, 20, or more piezoelectric elements. In some embodiments, the piezoelectric elements are coupled in series, with the distal end portion of the first piezoelectric element coupled to a fixation region within the housing, the proximal end portion of the last piezoelectric element coupled to the harvester mass, and the intermediate piezoelectric elements being coupled to neighboring piezoelectric elements at their respective proximal and distal end portions. At least some of the intermediate piezoelectric elements can be positioned at different sides of the first piezoelectric element, e.g., to allow the first piezoelectric element to be coaxial with and/or received within the lumen of the power assembly.

Additionally, although the embodiments of FIGS. 5A-6 illustrate piezoelectric elements coupled to each other via joints, in other embodiments, some or all of the piezoelectric elements can instead be a single monolithic component that is bent or otherwise formed into the desired configuration, such as an elongate metal strip with upper and lower piezoelectric layers. This approach can improve the reliability of the energy harvesting mechanism by obviating the need for joints, bonding, welding, adhesives, etc., to connect the piezoelectric elements to each other.

### EXAMPLES

The following examples are included to further describe some aspects of the present technology, and should not be used to limit the scope of the technology.

Example 1. A device comprising: a housing configured to be implanted within a patient; a power assembly positioned within the housing and comprising a power source; and an energy harvesting mechanism positioned within the housing and configured to charge the power source, wherein the energy harvesting mechanism comprises: a first piezoelectric element coupled to a fixation region within the housing, a second piezoelectric element coupled to a harvester mass, and a third piezoelectric element coupled in series between the first and second piezoelectric elements, wherein the second and third piezoelectric elements are positioned at different sides of the first piezoelectric element.

Example 2. The device of Example 1, wherein the second piezoelectric element is positioned proximate to a first side of the first piezoelectric element, and the third piezoelectric element is positioned proximate to a second side of the first piezoelectric element.

Example 3. The device of Example 2, wherein the first side is opposite the second side.

Example 4. The device of any one of Examples 1 to 3, wherein: the housing has a proximal end, a distal end, and a longitudinal axis extending from the proximal end to the distal end, and the first, second, and third piezoelectric elements each have a respective longitudinal axis that is aligned with the longitudinal axis of the housing.

Example 5. The device of any one of Examples 1 to 4, wherein the power assembly comprises a tubular structure having a lumen extending therethrough, and a portion of the first piezoelectric element is received within the lumen.

Example 6. The device of Example 5, wherein the second and third piezoelectric elements are outside of the lumen of the power assembly.

Example 7. The device of any one of Examples 1 to 6, wherein the energy harvesting mechanism further comprises a plurality of joints coupling the first, second, and third piezoelectric elements to each other.

Example 8. The device of Example 7, wherein the plurality of joints electrically couple the first, second, and third piezoelectric elements to each other.

Example 9. The device of Example 7 or 8, wherein the plurality of joints comprises: a first joint coupling the first piezoelectric element to the third piezoelectric element, and a second joint coupling the third piezoelectric element to the second piezoelectric element.

Example 10. The device of Example 9, wherein the second joint includes a recess, and a portion of the first piezoelectric element passes through the recess.

Example 11. The device of any one of Examples 1 to 8, wherein the energy harvesting mechanism further comprises: a fourth piezoelectric element coupled in series between the first and second piezoelectric elements, and a fifth piezoelectric element coupled in series between the first and second piezoelectric elements.

Example 12. The device of any one of Examples 1 to 11, wherein the energy harvesting mechanism has a resonant frequency within a range from 10 Hz to 30 Hz.

Example 13. A device comprising: a housing configured to be implanted within a patient; a tubular accumulator positioned within the housing, the tubular accumulator having a lumen extending therethrough; and an energy harvester positioned within the housing and configured to provide power to the tubular accumulator, wherein the energy harvester comprises: a first piezoelectric member coupled to a fixation region within the housing, the first piezoelectric member having a central longitudinal axis that is aligned with the lumen of the tubular accumulator, a second piezoelectric member coupled to a harvester mass, and a third piezoelectric member coupled in series between the first and second piezoelectric members.

Example 14. The device of Example 13, wherein a portion of the first piezoelectric member is received within the lumen of the tubular accumulator.

Example 15. The device of Example 13 or 14, wherein the fixation region is distal to the tubular accumulator.

Example 16. The device of any one of Examples 13 to 15, wherein the tubular accumulator has a central longitudinal axis extending through the lumen, and the central longitudinal axis of the first piezoelectric member overlaps the central longitudinal axis of the tubular accumulator.

Example 17. The device of Example 16, wherein the second and third piezoelectric members are spaced apart from the central longitudinal axis of the tubular accumulator.

Example 18. The device of any one of Examples 13 to 17, wherein the second and third piezoelectric members are positioned proximal to the lumen of the tubular accumulator.

Example 19. The device of any one of Examples 13 to 18, wherein the second and third piezoelectric members are positioned at different sides of the first piezoelectric member.

Example 20. The device of any one of Examples 13 to 19, wherein the energy harvesting mechanism further comprises a plurality of joints coupling the first, second, and third piezoelectric members to each other.

Example 21. The device of Example 20, wherein the plurality of joints electrically couple the first, second, and third piezoelectric members to each other.

Example 22. The device of Example 20 or 21, wherein the plurality of joints comprises: a first joint coupling the first piezoelectric member to the third piezoelectric member, and a second joint coupling the third piezoelectric member to the second piezoelectric member.

Example 23. The device of Example 22, wherein the second joint includes a recess, and a portion of the first piezoelectric member passes through the recess.

Example 24. The device of any one of Examples 13 to 21, wherein the energy harvesting mechanism further comprises: a fourth piezoelectric member coupled in series between the first and second piezoelectric members, and a fifth piezoelectric element coupled in series between the first and second piezoelectric members.

Example 25. The device of any one of Examples 13 to 24, wherein the energy harvesting mechanism is configured to produce power from physiological motion.

Example 26. A device comprising: a housing configured to be implanted within a patient; a power source positioned within the housing; and an energy harvesting mechanism positioned within the housing and configured to charge the power source, wherein the energy harvesting mechanism comprises: a first piezoelectric element coupled to a fixation region within the housing, a second piezoelectric element coupled to a harvester mass, and a third piezoelectric element coupled in series between the first and second piezoelectric elements, wherein the second and third piezoelectric elements are positioned at different sides of the first piezoelectric element.

Example 27. The device of Example 26, wherein the second piezoelectric element is positioned proximate to a first side of the first piezoelectric element, and the third piezoelectric element is positioned proximate to a second side of the first piezoelectric element.

Example 28. The device of Example 27, wherein the first side is opposite the second side.

Example 29. The device of any one of Examples 26 to 28, wherein: the housing has a proximal end, a distal end, and a longitudinal axis extending from the proximal end to the distal end, and the first, second, and third piezoelectric elements each have a respective longitudinal axis that is aligned with the longitudinal axis of the housing.

Example 30. The device of any one of Examples 26 to 29, wherein the power source comprises a tubular structure having a lumen extending therethrough, and a portion of the first piezoelectric element is received within the lumen.

Example 31. The device of Example 30, wherein the second and third piezoelectric elements are outside of the lumen of the power source.

Example 32. The device of any one of Examples 26 to 31, wherein the energy harvesting mechanism further comprises a plurality of joints coupling the first, second, and third piezoelectric elements to each other.

Example 33. The device of Example 32, wherein the plurality of joints electrically couple the first, second, and third piezoelectric elements to each other.

Example 34. The device of Example 32 or 33, wherein the plurality of joints comprises: a first joint coupling the first piezoelectric element to the third piezoelectric element, and a second joint coupling the third piezoelectric element to the second piezoelectric element.

Example 35. The device of Example 34, wherein the second joint includes a recess, and a portion of the first piezoelectric element passes through the recess.

Example 36. The device of any one of Examples 26 to 35, wherein the energy harvesting mechanism further comprises: a fourth piezoelectric element coupled in series between the first and second piezoelectric elements, and a fifth piezoelectric element coupled in series between the first and second piezoelectric elements.

Example 37. The device of any one of Examples 26 to 36, wherein the energy harvesting mechanism has a resonant frequency within a range from 10 Hz to 30 Hz.

### Conclusion

Although many of the embodiments are described above with respect to systems, devices, and methods for cardiac pacing, the technology is applicable to other applications and/or other approaches, such as other therapies involving implantable devices. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-6.

The embodiments of the present technology can be implemented, at least in part, in hardware, software, firmware or any combination thereof. For example, various embodiments can be implemented within one or more processors, including one or more microprocessors, DSPs, ASICs, FPGAs, or any other equivalent integrated or discrete logic circuitry, as well as any combinations of such components, embodied in programmers (e.g., physician or patient programmers), stimulators, or other devices. The terms “processor” or “processing circuitry” may generally refer to any of the foregoing logic circuitry, alone or in combination with other logic circuitry, or any other equivalent circuitry.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1.-15. (canceled)

16. A device comprising:
a housing configured to be implanted within a patient;
a power assembly positioned within the housing and comprising a power source; and
an energy harvesting mechanism positioned within the housing and configured to charge the power source, wherein the energy harvesting mechanism comprises:
a first piezoelectric element coupled to a fixation region within the housing,
a second piezoelectric element coupled to a harvester mass, and
a third piezoelectric element coupled in series between the first and second piezoelectric elements,
wherein the second and third piezoelectric elements are positioned at different sides of the first piezoelectric element.

17. The device of claim 16, wherein the second piezoelectric element is positioned proximate to a first side of the first piezoelectric element, and the third piezoelectric element is positioned proximate to a second side of the first piezoelectric element.

18. The device of claim 17, wherein the first side is opposite the second side.

19. The device of claim 16, wherein:
the housing has a proximal end, a distal end, and a longitudinal axis extending from the proximal end to the distal end, and
the first, second, and third piezoelectric elements each have a respective longitudinal axis that is aligned with the longitudinal axis of the housing.

20. The device of claim 16, wherein the power assembly comprises a tubular structure having a lumen extending therethrough, and a portion of the first piezoelectric element is received within the lumen.

21. The device of claim 20, wherein the second and third piezoelectric elements are outside of the lumen of the power assembly.

22. The device of claim 16, wherein the energy harvesting mechanism further comprises a plurality of joints coupling the first, second, and third piezoelectric elements to each other.

23. The device of claim 22, wherein the plurality of joints comprises:
a first joint coupling the first piezoelectric element to the third piezoelectric element, and
a second joint coupling the third piezoelectric element to the second piezoelectric element.

24. The device of claim 23, wherein the second joint includes a recess, and a portion of the first piezoelectric element passes through the recess.

25. The device of claim 16, wherein the energy harvesting mechanism has a resonant frequency within a range from 10 Hz to 30 Hz.

26. A device comprising:
a housing configured to be implanted within a patient;
a tubular accumulator positioned within the housing, the tubular accumulator having a lumen extending therethrough; and
an energy harvester positioned within the housing and configured to provide power to the tubular accumulator, wherein the energy harvester comprises:
a first piezoelectric member coupled to a fixation region within the housing, the first piezoelectric member having a central longitudinal axis that is aligned with the lumen of the tubular accumulator,
a second piezoelectric member coupled to a harvester mass, and
a third piezoelectric member coupled in series between the first and second piezoelectric members.

27. The device of claim 26, wherein a portion of the first piezoelectric member is received within the lumen of the tubular accumulator.

28. The device of claim 26, wherein the fixation region is distal to the tubular accumulator.

29. The device of claim 26, wherein the tubular accumulator has a central longitudinal axis extending through the lumen, and the central longitudinal axis of the first piezoelectric member overlaps the central longitudinal axis of the tubular accumulator.

30. The device of claim 29, wherein the second and third piezoelectric members are spaced apart from the central longitudinal axis of the tubular accumulator.

31. The device of claim 26, wherein the second and third piezoelectric members are positioned proximal to the lumen of the tubular accumulator.

32. The device of claim 26, wherein the second and third piezoelectric members are positioned at different sides of the first piezoelectric member.

33. The device of claim 26, wherein the energy harvesting mechanism further comprises a plurality of joints coupling the first, second, and third piezoelectric members to each other.

34. The device of claim 33, wherein the plurality of joints comprises:
a first joint coupling the first piezoelectric member to the third piezoelectric member, and
a second joint coupling the third piezoelectric member to the second piezoelectric member.

35. The device of claim 34, wherein the second joint includes a recess, and a portion of the first piezoelectric member passes through the recess.
